(19) United States
(12) Patent Application Publication
(10) Pub. No.: US 2022/0023134 A1
(43) Pub. Date: Jan. 27, 2022

(54) DEVICE AND METHOD FOR ASSISTING WITH MOBILIZING A JOINT

(71) Applicants: Lilas DEVILLE, Buxerolles (FR); Jérémy GASTEBOIS, Buxerolles (FR)

(72) Inventors: Lilas DEVILLE, Buxerolles (FR); Jérémy GASTEBOIS, Buxerolles (FR)

(21) Appl. No.: 17/276,986

(22) PCT Filed: Sep. 17, 2019
(86) PCT No.: PCT/FR2019/052158

§ 371 Date: Mar. 17, 2021

(51) Int. Cl. A61H 3/00; G16H 20/30; G16H 40/63; B25J 9/10; B25J 9/00

(52) U.S. Cl. CPC A61H 3/00; G16H 20/30; G16H 40/63; B25J 9/1045; A61H 2201/165; A61H 2201/149; A61H 2201/5002; A61H 2201/5007; B25J 9/0006

(57) ABSTRACT

Device (100) for assisting with mobilizing a joint (3) comprising a first and a second guiding element, the first flexible guiding element (102) being positioned on one of the segments (4), having a first pair of cable passages arranged on the side of the rear face (52) and on the side of the front face (54), the second flexible guiding element (104) being positioned on the other of the segments (5), having a first pair of cable passages, arranged on the side of the rear face (52) and on the side of the front face (54), the second guiding element further comprising a pair of anchoring points arranged on the side of the front face (52) and on the side of the rear face (54), a flexible support part on which a pair of actuators (M11, M12) associated with a pair of winders are arranged, to cause the associated winder to turn in the winding direction and the unwinding direction, and a pair of cables (C11, C12) extending from the winders, up to the anchoring points of the second guiding element, passing through the cable passages of the first pair of cable passages of the first and second guiding elements.

### TECHNICAL FIELD

The invention relates to the field of flexible exoskeleton devices for assisting with mobilising at least one joint of a body, and more particularly to that of cable-driven flexible exoskeletons for assisting with mobilising at least one joint. The invention also relates to a method using a device according to the invention.

### BACKGROUND OF THE INVENTION

From international application WO 2017/026943, a cable-driven device for assisting with mobilising a joint of a body, according to a flexion movement or an extension movement of the elbow, is known. The device has two cables, one connected to the posterior part of the forearm, the other connected to the anterior part of the forearm.

In the present description, the anatomy reference system is used, when the subject is in the standard human anatomical position, the so-called Paul Poirier position.

The device according to the prior art is not completely satisfactory.

This device may not be suitable for many people when it is tried to apply it to a joint such as that of the knee which connects the femur to the tibia and to the patella.

Indeed, some subjects can suffer from valgus or varus. In the case of the knee, varus corresponds to an outward rotation of the femur, which moves the knee joint laterally away resulting visually in bowlegs. In contrast, valgus is an inward rotation of the femur which brings the knee closer to the longitudinal axis, resulting in so-called “knock” knees. It is understood that applying the device according to the prior art to a person suffering from valgus or varus would result in generating a rotational torque on the ankle along the antero-posterior axis, which is not desired.

Furthermore, it is difficult to precisely determine the axis of rotation of the knee, which is not always colinear with the transverse axis. Also, the use of the device according to the prior art could result in applying a torque to the knee joint which has a component that is not colinear with the axis of the joint, which could be painful or even dangerous. To ascertain the position of the axis of rotation of a joint, biomechanics experts may use motion capture techniques, which make it possible to achieve reasonable precisions, in exchange for complex use. These techniques therefore cannot be envisaged for an exoskeleton that is intended to be easily adaptable. Furthermore, the position of the axis of rotation of a joint can be displaced during movement, this is for example the case of the knee.

Within the context of the knee joint, it is therefore understood that the device proposed by the prior art is not fully satisfactory for some people.

In fact, the device according to the prior art is likewise not fully satisfactory in the context of the use thereof for an elbow. Indeed, besides the flexion and extension movements of the forearm in relation to the forearm, elbow movements enable a second degree of freedom which is pronosupination, which is the rotation of the forearm about the longitudinal axis thereof.

Further joints are concerned. For example, the ankle joint, which connects the leg and the foot, can have an eversion, which corresponds to a lateral outward rotation. In the case of the heelbone (calcaneum), the sole of the foot is laterally oriented. Conversely, inversion is a medial inward rotation. The sole of the foot is then medially oriented. Further foot movements are possible, due to the specific joints of the foot. The positioning of a foot in relation to a leg solely by means of two cables is therefore very approximate.

Moreover, in the human body, the muscles responsible for movements operate in pairs, and are referred to as agonist and antagonist. The femoral quadriceps and the hamstring muscles for the knee or the biceps and the triceps for the elbow are two examples of these pairs of muscles.

When the agonist muscle is contracted to make a movement, for example a flexion of the knee, its antagonist muscle is stretched so as not to impede the movement. The roles are reversed when the opposite movement is made. In the context of the knee, the quadriceps contracts and the hamstring muscles stretch during knee extension. In contrast, the quadriceps stretches and the hamstring muscles contract during knee flexion.

The device according to the prior art is configured to only use a single motor to actuate the two cables, in alternation. In other words, only a single direction of movement of the arm can be actuated at once when a device according to the prior art is used. This gives rise to a pretension problem of the cable which is not actuated. Moreover, it is then not possible to apply a force along two opposite directions. In conclusion, it is only possible to use this type of device for a limited number of positional guidances of the joint.

The document U.S. Pat. No. 1,308,675 A, which only describes a pair of cables which separate into two above the hips, is also known. The two cables passing in front of the knee are controlled simultaneously by the actuation unit located in the back. The two cables located behind the knee are actuated simultaneously passively by springs located on the front of the torso. The direction of the joint torque cannot be controlled.

The document WO 2016/089466 A2, WO 2018/135402 A1, WO 2014/109799 A1, which only describe one cable (or one cable split into two) passing in front of the hip and behind the knee, or at the hip and the ankle and has the same drawbacks as U.S. Pat. No. 1,308,675 A, are also known.

### DESCRIPTION OF THE INVENTION

An aim of the invention is particularly that of remedying all or some of the drawbacks cited above.

According to a first aspect of the invention, a device for assisting with mobilising a joint connecting an upper segment (i.e. a bone) and a lower segment of a person is proposed, including:
a first flexible guiding element configured to be positioned on or in the vicinity of one of the segments, the first guiding element having a first pair of cable passages, one cable passage, respectively the other cable passage of the first pair of cable passages of the first guiding element being disposed on the side of the posterior face, respectively on the side of the anterior face, of said segment when the person wears the first guiding element,
a second flexible guiding element configured to be positioned on or in the vicinity of the other of the segments, the second guiding element having a first pair of cable passages, one cable passage, respectively the other cable passage of the first pair of cable passages being disposed on the side of the posterior face, respectively on the side of the anterior face, of said segment when the person wears the second guiding element, the second guiding element further including a pair of anchoring points, one anchoring point, respectively the other anchoring point, of the pair of anchoring points being disposed on the side of the anterior face, respectively on the side of the posterior face, of said segment,
a flexible support part arranged to be worn by the person and whereon a pair of actuators and a pair of winders are disposed, each of the actuators of the pair of actuators being associated with a winder of the pair of winders and being arranged to turn the associated winder in the winding direction and the unwinding direction,
a pair of cables including a first cable, respectively a second cable extending from one of the winders, respectively the other of the winders, of the pair of winders, to one of the anchoring points, respectively the other of the anchoring points, of the flexible anchoring element, passing, on one hand, through one cable passage, respectively the other cable passage, of the first pair of cable passages of the first guiding element and, on the other, through one cable passage, respectively the other cable passage, of the first pair of cable passages of the second guiding element,
a controller configured to control each of the actuators of the pair of actuators independently.

Advantageously, the controller is configured to control the actuators of the pair of actuators according to a predetermined control law.

Preferably, the first flexible guiding element further includes a second pair of cable passages, one cable passage, respectively the other cable passage of said second pair of cable passages of the first guiding element being disposed on the side of the posterior face, respectively on the side of the anterior face, of said segment when the person wears the first guiding element,

Also preferably, the second flexible guiding element further includes a second pair of cable passages, one cable passage, respectively the other cable passage of said second pair of cable passages of the second guiding element being disposed on the side of the posterior face, respectively on the side of the anterior face, of said segment when the person wears the second guiding element, the second guiding element optionally further including a second pair of anchoring points, one anchoring point, respectively the other anchoring point, of the second pair of anchoring points being disposed on the side of the anterior face, respectively on the side of the posterior face, of the lower segment when the person wears the second guiding element,

Also preferably, the device further includes a second pair of actuators and a second pair of winders, each of the actuators of the second pair of actuators being associated with a winder of the pair of winders and being arranged to turn the associated winder in the winding direction and the unwinding direction, the second pairs of actuators and winders being attached to the flexible support part, and a second pair of cables including a first cable, respectively a second cable, extending from one of the winders, respectively the other of the winders, of the second pair of winders, up to one of the anchoring points, respectively the other of the anchoring points, of the second guiding element, by passing, on one hand, through one cable passage, respectively the other cable passage, of the second pair of cable passages of the first guiding element and, on the other, through one cable passage, respectively the other cable passage, of the second pair of cable passages of the second guiding element, the controller being further configured to control, independently, each of the actuators of the second pair of actuators.

Each of the cables of the first and/or, where applicable, the second, pair of cables can be a flexible cable capable of sliding in a flexible sheath. The flexible cable can be of the type of the line used in aerial sports such as paragliding, for questions of ease of use. This type of cord withstands very large loads, is virtually not deformed and is easy to use. It has the advantage of being very flexible, and can be wound easily around reels of small diameter. The flexible cable can also be made of twisted steel, of a judiciously selected diameter.

The two ends of each cable can be provided with a crimped or screwed end piece, and are connected, respectively, to one of the winders and the anchoring point thereof. The sheath can be composed of a joined spire spring, and can be wrapped with a cotton or plastic sheath.

When the first flexible guiding element and the second flexible guiding element are disposed on the person, each of the cables has a remarkable point, defined as the closest point to an estimated position of the axis of rotation of the joint.

According to one option, the direction formed by the remarkable points of the first pair of cables, and that formed by the two remarkable points of the second pair of cables are substantially orthogonal.

Advantageously, the directions form an angle of 45° with the transverse axis and the anteroposterior axis, when the person wears the anchoring element (in other words, left right front back).

Even more advantageously, it is proposed to use more than two pairs of cables. Actuation redundancy is then obtained. In the context of large joints, such as the back, for example, actuation redundancy makes it possible to use a greater number of less powerful motors and furthermore to better distribute the load on the patient.

According to a specificity, the device according to the first aspect of the invention includes a means for determining the orientation of the upper segment and/or the lower segment with respect to the longitudinal axis.

Preferably, the device according to the first aspect of the invention further includes a means for determining the joint torque.

According to a specificity, the device according to the first aspect of the invention includes a means for determining the orientation of the upper segment and/or the lower segment with respect to the longitudinal axis.

The means for determining the joint torque and the orientation of the segments with respect to the longitudinal axis (orientation with respect to gravity) can be embodied using a means for determining the two angles formed between the upper segment and the lower segment.

By way of example, the means for determining the two angles can be embodied by means of two bidirectional inclinometers, one placed on the upper segment and the other on the lower segment. The two inclinometers make it possible to measure the angles of the upper segment and of the lower segment in the sagittal and transverse planes. These angle sensors make it possible to measure the geometric arrangement of the segments and to infer the static gravity torque on the knee therefrom. The measurement of the geometric arrangement of the segments enables an estimated reconstruction of the position of the axis of rotation, and the two pairs of cables can be actuated in order to generate a torque which is the most aligned possible with this reconstruction of the axis of rotation.

The means for determining the joint torque can include inertial units. A determination of the joint torque can also take dynamic phenomena into account. It is possible to measure the accelerations to which each of the segments is subjected, and thus be able to infer the forces to which they are subjected therefrom. In this way, it is possible to determine the torque exerted on the joint, which is the sum of the gravity torque and of all the other factors liable to contribute (inertia, etc.). The gravity torque can be inferred from the position, that can be obtained by two successive integrations of the accelerations. The use of inertia units enables a better approximation of the reconstruction of the axis of rotation.

According to an embodiment, it is selected to use force sensors placed at the end of each of the cables, and to measure the difference between the cable tension measured by these sensors and the torque developed by the actuators, for example measured using the current in the motors, which determines a measurement of the exoskeleton wearer's “intention”. Indeed, the actuator torque represents the torque supplied by the exoskeleton and the measurement of the sensors makes it possible to determine whether the person pulls on some cables or relaxes others, which makes it possible to infer the progression of the movement.

By way of example, the device according to the first aspect of the invention, or one or more of the enhancements thereof, includes a means for detecting a bearing of the lower segment on a floor or a frame.

Also by way of example, the device according to the first aspect of the invention, or one or more of the enhancements thereof, has a power transmission chain between a motor and an anchoring point which includes a hoist.

According to a second aspect of the invention, a method, preferably non-therapeutic, for assisting, preferentially in the context of leisure or sports activity or applications for assisting the person at home (for example, intended for the elderly, or those having reduced mobility), for mobilising a joint connecting an upper segment and a lower segment, including a device for mobilising the joint according to the first aspect of the invention, or one or more of the enhancements thereof, is proposed.

Force sensors can be used to control the device according to the invention.

According to one option, the assistance method can further comprise a step of estimating the joint torque based on information from sensors equipping the device, and a step of computing and applying an assistance torque, corresponding to a predetermined assistance percentage.

Advantageously, the assistance method can further comprise a step of filtering, for example of low-pass type, the information from the sensors.

The assistance method can include a filtering step, wherein the control law of the device is determined using a predetermined trajectory.

The assistance method can include a tracking of the progression of the position deviation in absolute values between, on one hand, the actual position of the lower segment at a given time, and, on the other, the position setpoint of the determined trajectory at said given time.

The assistance method can include an application by the actuators of the device, on one hand of a reactive force when said deviation increases and, on the other, of an active force when said deviation decreases.

According to a third aspect of the invention, a computer program product, loadable directly in the internal memory of a computer, comprising software code portions for executing the steps of the visualisation method according to the second aspect of the invention, or one or more of the enhancements thereof, when said program is executed on a computer, is proposed.

The computer program product can include means for setting the assistance method, for example by means of a user interface, preferably displayed on a computer or smartphone screen.

The settings can for example be assistance intensity, operating mode settings.

### DESCRIPTION OF THE FIGURES

Further advantages and specificities of the invention will emerge on studying the detailed description of implementations and embodiments, which are in no way restrictive, in relation to the appended drawings wherein:

FIG. 1 schematically represents, three perspective and partial views of a right leg, respectively from the left, the rear, and the right, equipped with a device according to an embodiment of the invention,

FIG. 2 schematically represents the positioning of remarkable cable passage points of the device represented in FIG. 1, along a transverse plane; it is a cross-sectional view of the knee (and of the device) along the transverse plane.

### DESCRIPTION OF EMBODIMENTS

As the embodiments described hereinafter are in no way restrictive, alternative embodiments of the invention only comprising a selection of described features, subsequently isolated from the other described features may particularly be considered, if this selection of features is sufficient to provide a technical advantage or to differentiate the invention with respect to the prior art. This selection comprises at least one feature, preferably functional with no structural details, or with merely a portion of the structural details if this portion only is sufficient to provide a technical advantage or to differentiate the invention with respect to the prior art.

In the figures, an element appearing in several figures retains the same reference.

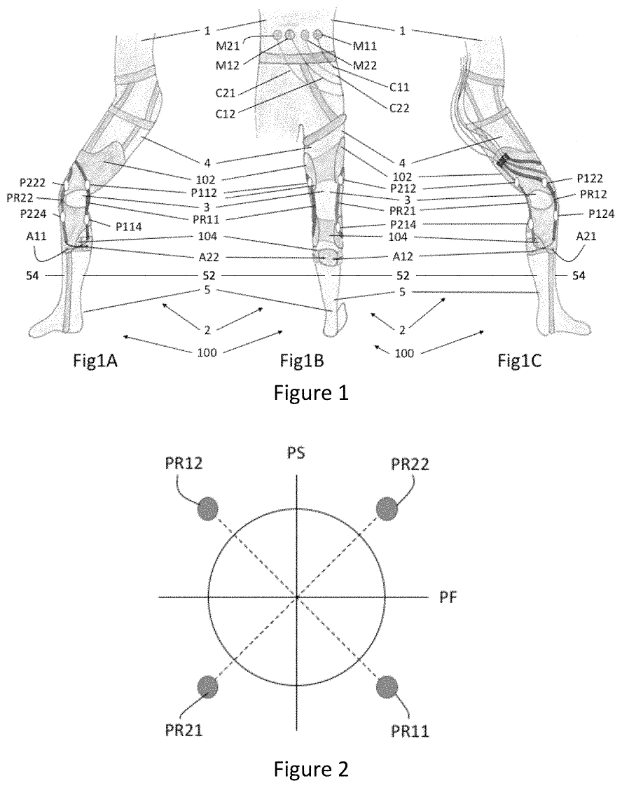

FIGS. 1A, 1B and 1C represent a part of a person 1, and particularly a right leg 2, respectively according to a left side view, a rear view and a right side view.

The leg 2 is equipped with a device 100 for assisting with mobilising a joint 3 connecting an upper segment 4 and a lower segment 5.

According to the embodiment illustrated, the joint 3 is a right knee, the upper segment 4, respectively the lower segment 5, being formed from a femur, respectively from the tibia and the patella.

The device 100 includes:
a first flexible guiding element 102 configured to be positioned on or in the vicinity of the upper segment 4 of the person, when the person wears the first guiding element,
a second flexible guiding elements 104 configured to be positioned on or in the vicinity of the lower segment 5 of the person 1, when the person wears the second guiding element,
a flexible support part 106 (not shown) arranged to be worn by the person 1.

The first guiding element 102 includes:
a first pair of cable passages, one cable passage P112, respectively the other cable passage P122, of the first pair of cable passages being disposed on the side of the posterior face 52, respectively on the side of the anterior face 54, of said segment when the person wears the first guiding element,
a second pair of cable passages, one cable passage P212, respectively the other cable passage P222, of the second pair of cable passages being disposed on the side of the posterior face 52, respectively on the side of the anterior face 54, of said segment when the person wears the first guiding element,

The second flexible anchoring guiding element 104 includes:
a first pair of cable passages, one cable passage P114, respectively the other cable passage P124, of the first pair of cable passages being disposed on the side of the posterior face 52, respectively on the side of the anterior face 54, of said segment when the person wears the second guiding element,
a second pair of cable passages, one cable passage P214, respectively the other cable passage P224, of the second pair of cable passages being disposed on the side of the posterior face 52, respectively on the side of the anterior face 54, of said segment when the person wears the first guiding element,
a first pair of anchoring points, one anchoring point A11, respectively the other anchoring point A12, of the first pair of anchoring points being disposed on the side of the anterior face 52, respectively on the side of the posterior face 54, of said segment when the person wears the second guiding element 104,
a second pair of anchoring points, one anchoring point A21, respectively the other anchoring point A22, of the second pair of anchoring points being disposed on the side of the anterior face 52, respectively on the side of the posterior face 54, of said segment when the person wears the second guiding element 104.

Two pairs of actuators M11, M12 and M21, M22 of the gear motor type and two pairs of winders (not shown) are disposed on the flexible support part 106, each of the actuators of the pair of actuators being associated with a winder of the pair of winders and being arranged to turn the associated reel type winders along the winding direction and the unwinding direction.

The device 100 further includes:
two pairs of cables C11, C12 and C21, C22,
a controller (not shown) configured to control each of the actuators of the pair of actuators.

The first pair of cables C11, C12 includes a first cable C11, respectively a second cable C12, extending from the winder connected to the actuator M11, respectively the other winder connected to the actuator M12, of each of the pairs of winders, to one of the anchoring points A11, respectively the other of the anchoring points A12, of the second guiding element 104, passing, on one hand, through one cable passage P112, respectively the other cable passage P122, of the first pair of cable passages of the first guiding element 102 and, on the other, through one cable passage P114, respectively the other cable passage P124, of the first pair of cable passages of the second guiding element 104.

The second pair of cables C21, C22 includes a first cable C21, respectively a second cable C22, extending from the winder connected to the actuator M21, respectively the other winder connected to the actuator M22, to one of the anchoring points A21, respectively the other of the anchoring points A22, of the second guiding element 104, passing, on one hand, through one cable passage P212, respectively the other cable passage P222, of the second pair of cable passages of the first guiding element 102 and, on the other, through one cable passage P214, respectively the other cable passage P224, of the second pair of cable passages of the second guiding element 104.

When the first flexible guiding element and the second flexible guiding element are disposed on the person, each of the cables has a remarkable point, defined as the closest point to the axis of rotation of the joint.

FIG. 2 illustrates a cross-section of the knee of the right leg viewed from below, in the transverse plane. The sagittal plane PS and the frontal plane PF are represented therein, as well as the four remarkable points PR11, PR12, PR21 and PR22, respectively, through which the cables C11, C12, C21 and C22, respectively, pass.

As illustrated in FIG. 2, the directions formed are substantially orthogonal. The superposition of the torques produced by the two pairs of cables makes it possible to generate any torque perpendicular to the transverse plane.

More specifically, the directions formed by the remarkable points PR11, PR12 of the first pair of remarkable points, respectively the two remarkable points PR21, PR22 of the second pair of remarkable points, form an angle of 45° with the transverse axis and the anteroposterior axis, when the person wears the anchoring element. This arrangement of the cables is more comfortable than if two cables were disposed in the frontal plane, which would be particularly uncomfortable. Indeed, one of the two cables of a pair would be located between the legs, thus impeding walking and one of the two cables of the other pair would be located behind the knee, which would be uncomfortable for bending the knee completely.

The positioning of the cable passages is used to ensure a distance between each cable and an estimated position of the centre of the joint. The passages are also used in such a way that the free portions of the stretched cables are distributed as regularly as possible around the circumference of the knee. The cable can be inserted into Bowden type sheaths in order to minimise the deformation of the tensioned cables. Cable passage positioning imprecisions can be compensated by means of a robust control law.

The controller is configured to control the actuators of the pair of actuators according to a predetermined control law.

Use of a Device for Assisting with Mobilising the Joint

With a device according to the invention, according to a first option, a method for assisting with movement is proposed, including:
a step of estimating the joint torque based on information from the sensors (static or dynamic)
a step of computing and applying an assistance torque, corresponding to an assistance percentage selected in advance, for example by a doctor.

According to one option, a non-medicinal solution is thus proposed, making it possible to restore or retain physical activity and thus reinforce the joints affected by providing a level of assistance adapted to the case of the patient affected. It is thus possible to delay the use of medical treatment or reduce it, and delay the use of prostheses in the most severe cases.

According to one option, a solution is thus proposed for providing assistance with mobility in the context of leisure or sports activities, as well as on a day-to-day basis for persons having mobility problems.

According to a second option, a method for learning a movement is proposed, wherein the control law of the device is determined using a predetermined trajectory.

The method comprises tracking of the progression of the position deviation in absolute values between, on one hand, the actual position of the lower segment at a given time and, on the other, the position set-point of the determined trajectory at said given time, and an application by the actuators of the device, on one hand of a reactive force when said deviation increases and, on the other of an active force when said deviation decreases.

More specifically, the method comprises:
a step of recording a movement to be learnt,
a step of generating the force field corresponding to the movement to be learnt,
a step of estimating the absolute value of the position deviation,
   if the absolute value of the position deviation increases, generating an additional force field opposing the person's movement,
   if the absolute value of the position deviation decreases or becomes zero, generating a force field, optionally zero, assisting with, or not impeding, the movement.

For the step of recording the movement to be learnt, equipping a person mastering the movement to be learnt with a device according to the invention is envisaged. It would then be in “recording” mode and would passively measure the trajectory followed. This trajectory would be used to create a three-dimensional force field, the function of which would be to encourage the “right” movement and discourage the “wrong” movement. When the person equipped with the exoskeleton and needing to learn the movement deviates from the trajectory to follow, the exoskeleton counters a resistance increasing with the deviation from the trajectory. On the contrary, when the individual follows a close trajectory to the trajectory to be followed, the exoskeleton exerts no resistance, or even proposes an adapted resistance.

The same principle could be used for rehabilitation applications, placing an emphasis on the assistance provided to the patient.

Moreover, in addition to guiding movement to obtain a predetermined trajectory, for example a trajectory according to a walking movement, the method could guide to obtain a movement in the context of sports activities: gymnastics ring exercise, golf movement, swimming movement.

### Elimination of Tremors

With a device according to the invention, it is also possible to propose a method for reducing tremors.

Some diseases such as Parkinson's disease cause uncontrolled tremors in those affected. It can be envisaged to program the controller of the device according to the invention with a similar assistance to that described within the scope of the first option, and to add a well-adjusted low-pass filter thereto for filtering the subject's parasitic tremors.

Obviously, the invention is not limited to the examples described above and numerous adaptations can be made to these examples without leaving the scope of the invention. Furthermore, the different features, forms, alternative embodiments and embodiments of the invention can be associated with one another according to various combinations insofar as they are not incompatible or exclusive in relation to one another.

1. Device (100) for assisting with mobilising a joint (3) connecting an upper segment (4) and a lower segment (5) of a person (1), including:
a first flexible guiding element (102) configured to be positioned on or in the vicinity of one of the segments (4), the first guiding element having a first pair of cable passages attached to said first guiding element, one cable passage (P112), respectively the other cable passage (P122), of the first pair of cable passages being disposed on the side of the posterior face (52), respectively on the side of the anterior face (54), of said segment when the person wears the first guiding element,
a second flexible guiding element (104) configured to be positioned on or in the vicinity of the other of the segments (5), the second guiding element having a first pair of cable passages attached to said second guiding element, one cable passage (P114), respectively the other cable passage (P124), of the first pair of cable passages being disposed on the side of the posterior face (52), respectively on the side of the anterior face (54), of said segment when the person wears the second guiding element, the second guiding element further including a pair of anchoring points, one anchoring point (A11), respectively the other anchoring point (A12), of the pair of anchoring points being disposed on the side of the anterior face (54), respectively on the side of the posterior face (52), of said segment, when the person wears the second guiding element,
a flexible support part arranged to be worn by the person and whereon a pair of actuators (M11, M12) and a pair of winders are disposed, each of the actuators of the pair of actuators being associated with a winder of the pair of winders and being arranged to turn the associated winder in the winding direction and the unwinding direction,
a pair of cables (C11, C12) including a first cable (C11), respectively a second cable (C12) extending from one of the winders, respectively the other of the winders, of the pair of winders, to one of the anchoring points, respectively the other of the anchoring points, of the second guiding element, passing, on one hand, through one cable passage, respectively the other cable passage, of the first pair of cable passages of the first guiding element and, on the other, through one cable passage, respectively the other cable passage, of the first pair of cable passages of the second guiding element,
a controller configured to control each of the actuators of the pair of actuators independently.

2. Device according to claim 1, wherein the controller is configured to control the actuators of the pair of actuators according to a predetermined control law.

3. Device according to claim 2, wherein:
the first flexible guiding element (102) further includes a second pair of cable passages attached to said first guiding element, one cable passage (P212), respectively the other cable passage (P222), of said second pair of cable passages of the first guiding element being disposed on the side of the posterior face (52), respectively on the side of the anterior face (54), of said segment when the person wears the first guiding element,
the second flexible guiding element (104) further includes a second pair of cable passages attached to said second guiding element, one cable passage (P214), respectively the other cable passage (P224), of said second pair of cable passages of the second guiding element being disposed on the side of the posterior face (52), respectively on the side of the anterior face (54), of said segment when the person wears the second guiding element, the second guiding element further including a second pair of anchoring points, one anchoring point (A21), respectively the other anchoring point (A22), of the second pair of anchoring points being disposed on the side of the anterior face (54), respectively on the side of the posterior face (52), of the lower segment (5) of the person (1) when the person wears the second guiding element, the device further including:
a second pair of actuators (M21, M22) and a second pair of winders, each of the actuators of the second pair of winders being associated with a winder of the second pair of winders and being arranged to turn the associated winder along the winding direction and the unwinding direction, the second pairs of actuator and winder being attached to the flexible support part,
a second pair of cables (C21, C22) including a first cable (C21), respectively a second cable (C22) extending from one of the winders, respectively the other of the winders, of the second pair of winders, to one of the anchoring points, respectively the other of the anchoring points, of the second guiding element, passing, on one hand, through one cable passage, respectively the other cable passage, of the second pair of cable passages of the first guiding element and, on the other, through one cable passage, respectively the other cable passage, of the second pair of cable passages of the second guiding element,
a controller being furthermore configured to control each of the actuators of the second pair of actuators independently.

4. Device according to claim 1, wherein, when the first anchoring element and the second anchoring element are disposed on the person, each of the cable has a remarkable point, determined as the closest point to an estimated position of the axis of rotation of the joint, and the direction formed by the two remarkable points (PR11, PR12) of the first pair of cables and the direction formed by the two remarkable points (PR21, PR22) of the second pair of cables are substantially orthogonal.

5. Device according to claim 4, wherein, the directions form an angle of 45° with the transverse axis and the anteroposterior axis, when the person wears the anchoring element.

6. Device according to claim 1, including more than two pairs of cables.

7. Device according claim 1, including a means for determining the joint torque.

8. Device according to claim 1, including a means for determining the orientation of the upper segment and/or the lower segment with respect to the longitudinal axis.

9. Device according to claim 1, including a means for detecting a bearing of the lower segment on a floor or a frame.

10. Device according to claim 1, wherein a power transmission chain between a motor and an anchoring point includes a hoist.

11. Method (100) for controlling an assistance device according to claim 1.

12. Method according to claim 11, further including a step of estimating the joint torque based on information from sensors equipped the device, and a step of computing and applying an assistance torque, corresponding to a predetermined assistance percentage.

13. Method according to claim 12, further including a step of low-pass type filtering of the information from the sensors.

14. Method according to claim 13, when the device for mobilising the joint is embodied according to claim 2, wherein the control law of the device is determined using a predetermined trajectory.

15. Method according to claim 14, including tracking of the progression of the position deviation in absolute values between, on one hand, the actual position of the lower segment at a given time and, on the other, the position set-point of the determined trajectory at said given time, and an application by the actuators of the device, on one hand of a reactive force when said deviation increases and, on the other of an active force when said deviation decreases.

16. Computer program product, loadable directly in the internal memory of a computer, comprising software code portions for executing the steps of an assistance method according to claim 1, when said program is executed on a computer.
